(19) United States
(12) Patent Application Publication
(10) Pub. No.: US 2022/0273495 A1
(43) Pub. Date: Sep. 1, 2022

(54) Multi-Layer Wound Care Device Having Absorption and Fluid Transfer Properties

(71) Applicant: Milliken & Company, Spartanburg, SC (US)

(72) Inventors: Rajib Mondal, Greer, SC (US); Matthew I. Foote, Spartanburg, SC (US); Cristina M. Acevedo, Greer, SC (US); Geoffrey R. Haas, Spartanburg, SC (US)

(21) Appl. No.: 17/747,480

(22) Filed: May 18, 2022

Related U.S. Application Data
(62) Division of application No. 16/442,594, filed on Jun. 17, 2019.
(60) Provisional application No. 62/691,660, filed on Jun. 29, 2018.

(51) Int. Cl. A61F 13/00; A61L 15/20; A61L 15/44

(52) U.S. Cl. CPC A61F 13/00063; A61L 15/20; A61L 15/44; A61F 2013/00234; A61F 2013/00238; A61F 2013/00314; A61F 2013/530664

(57) ABSTRACT

This disclosure relates to a multi-layer wound care device having absorption and fluid transfer properties. The wound care device contains capillary force one-way pumps that are capable of transporting fluid, such as wound exudate, away from a wound site to the opposite side of the wound care device, which functions as a segregated fluid reservoir. This fluid transport mechanism generally aids in reducing wound maceration by removing excess wound fluid and the protease enzymes and infectious bacteria contained within the wound fluid. The wound care device performs this function, often times for multiple days, without the loss of the physical integrity of the wound care device. In addition to providing a uni-directional fluid transport mechanism, the wound care device provides improved absorption properties.

### CROSS-REFERENCE TO RELATED APPLICATIONS

This application claims priority to and is a divisional of U.S. patent application Ser. No. 16/442,594, entitled “Multi-Layer Wound Care Device Having Absorption and Fluid Transfer Properties,” which was filed on Jun. 17, 2019, which claims priority to U.S. Patent Application Ser. No. 62/691,660, entitled “Multi-Layer Wound Care Device Having Absorption and Fluid Transfer Properties,” which was filed on Jun. 29, 2018, both of which are entirely incorporated by reference herein.

### TECHNICAL FIELD

This disclosure relates to a multi-layer wound care device having absorption and fluid transfer properties. The wound care device contains capillary force one-way pumps that are capable of transporting fluid, such as wound exudate, away from a wound site to the opposite side of the wound care device, which functions as a segregated fluid reservoir. This fluid transport mechanism generally aids in reducing wound maceration by removing excess wound fluid and the protease enzymes and infectious bacteria contained within the wound fluid. The wound care device performs this function, often times for multiple days, without the loss of the physical integrity of the wound care device. In addition to providing a uni-directional fluid transport mechanism, the wound care device provides improved absorption properties.

In one aspect, the wound care device is comprised of a first fabric layer having a knit construction and comprising polyester fiber primarily present on the wound contact surface and lyocell fiber primarily present on the fluid reservoir surface. A third fiber, such as an elastomeric polyurethane known by the tradename Lycra®, may also be included in order to provide some amount of elasticity to the wound care device. The wound care device provides a one-way directional flow of fluid away from the wound and into the lyocell fluid reservoir.

### BACKGROUND

In the medical field, and in the area of wound care particularly, it is well-established that many factors, including the amount of moisture present at a wound site, affects how quickly a wound will heal. Generally speaking, having an excessive amount of moisture present at a wound site, especially when combined with the warm environment provided by the body, leads to undesirable bacteria growth and production of protease enzymes in the wound. Such growth can cause further damage to healthy cells and delay the healing process. However, insufficient moisture at the wound site can cause eschar (scab) formation and scarring and may cause the wound care device, or medical dressing, to adhere to the wound. If the dressing adheres to the wound, subsequent removal of the dressing may cause undue discomfort to the patient as well as disrupt newly granulated tissue. Infection of the wound may also be compounded when a medical dressing is removed and portions of the dressing remain behind in the wound itself, particularly if the dressing is already colonized with pathogenic microbes. Thus, it is important that the dressing maintains its physical integrity when exposed to stress, such as during removal from the wound, in order to prevent additional complications and delays in healing.

The wound care device of the present invention takes advantage of a unique textile fabric construction which effectively moves fluid away from the wound and provides improved absorption properties to the device. Both of these features promote and improve the healing process. The differentiation that exists in a wound care device having a hydrophobic fiber on the wound contact side of the device and a hydrophilic fiber on the fluid reservoir side of the device creates a unique one-way, directional flow of fluid and contaminants away from the wound. In addition, the incorporation of lyocell in the wound care device greatly enhances the absorption properties of the wound care device.

A further feature of the wound care device of the present invention is that the device may also contain a topical coating of an antimicrobial agent such as silver. It is known that placing surface-available silver in contact with a wound allows the silver to enter the wound and become absorbed by undesirable bacteria and fungi that grow and prosper in the warm, moist environment of the wound site. Once absorbed, the silver material kills microbes, resulting in treatment of infected wounds or the prevention of infection in at-risk wounds. Methods of topically applying a silver-based antimicrobial finish to textile substrates are described, for example, in commonly assigned U.S. Pat. Nos. 6,584,668; 6,821,936; and 6,946,433 and in commonly assigned U.S. patent application Ser. Nos. 09/586,081; 09/589,179; 10/307,027; and 10/306,968. All of these patents and patent applications are hereby incorporated by reference. Details of many of these processes will be discussed below.

The present disclosure addresses and overcomes the problems described above. Whereas, historically, a gauze or foam medical dressing has been applied to a wound with at least some intent on absorbing fluids, the present disclosure describes a wound care device capable of creating a one-way, directional flow of fluid and contaminants away from the wound, without detrimentally causing excessive dryness of the wound, and improved absorption properties of the wound care device. The wound care device may additionally provide desired release of silver to the wound site for antimicrobial efficacy and, because of its unique construction, maintains its physical integrity when exposed to stress during ordinary use of the wound care device.

For these reasons and others that will be described herein, the present wound care device having unique fluid management properties and improved absorption features represents a useful advance over the prior art.

### BRIEF SUMMARY

In one aspect, the invention relates to a wound care device comprising: a first layer of fabric having a wound contact surface and a wound fluid reservoir surface, wherein the first layer of fabric contains lyocell fibers; a first hotmelt adhesive; and a second layer of fabric; wherein the first hotmelt adhesive is disposed between the first layer of fabric and the second layer of fabric; and wherein the first hotmelt adhesive is disposed on the wound fluid reservoir surface of the first layer of fabric; and wherein the wound care device transports wound fluid uni-directionally from the wound contact surface to the wound fluid reservoir surface upon exposure to a wound.

In another aspect, the invention relates to a wound care device comprising: a first layer of fabric having a wound contact surface and a wound fluid reservoir surface, wherein the first layer of fabric contains lyocell fibers; a second layer of fabric, wherein the second layer of fabric has a wound facing surface and a non-wound facing surface; and at least one joining mechanism; wherein the at least one joining mechanism is in direct physical contact with the first layer of fabric and the second layer of fabric, and wherein the at least one joining mechanism is present on the wound contact surface of the first layer of fabric and on the non-wound facing surface of the second layer of fabric; and wherein the wound care device transports wound fluid uni-directionally from the wound contact surface to the wound fluid reservoir surface upon exposure to a wound.

In a further aspect, the invention relates to a wound care device comprising: a first layer of fabric having a wound contact surface and a wound fluid reservoir surface, wherein the first layer of fabric contains lyocell fibers; a second layer of fabric, wherein the second layer of fabric contains lyocell fibers; and at least one joining mechanism, wherein the at least one joining mechanism joins the first layer of fabric to the second layer of fabric; and wherein the wound care device transports wound fluid uni-directionally from the wound contact surface to the wound fluid reservoir surface upon exposure to a wound.

In a further aspect, the invention relates to a method for managing moisture at a wound site comprising the steps of: (a) providing a wound care device comprising: (i) a first layer of fabric having a wound contact surface and a wound fluid reservoir surface, wherein the first layer of fabric contains lyocell fibers; (ii) a second layer of fabric, wherein the second layer of fabric contains lyocell fibers; and (iii) at least one joining mechanism, wherein the at least one joining mechanism joins the first layer of fabric to the second layer of fabric; and wherein the wound care device transports wound fluid uni-directionally from the wound contact surface to the wound fluid reservoir surface upon exposure to a wound site; (b) placing the wound contact surface of the wound care device in contact with the wound site; and (c) allowing the wound care device to transport wound fluid uni-directionally from the wound contact surface to the wound fluid reservoir surface.

### BRIEF DESCRIPTION OF THE DRAWINGS

FIG. 1 is a plan view of a laid-in fabric suitable for use as the fluid transport layer of a wound care device according to the invention.

FIG. 2 is a schematic representation of a two-layer wound care device with stitch bonding.

FIG. 3 is a schematic representation of a three-layer wound care device with stitch bonding.

FIG. 4 is a schematic representation of a wound care device comprised of two layers of fabric joined with hotmelt adhesive.

FIG. 5A is a schematic representation of a wound care device comprised of three layers of fabric joined with hotmelt adhesive.

FIG. 5B is the same as FIG. 5A, except that the wound care device has been inverted for illustrative purposes and a border adhesive layer has been included.

FIG. 6 is a bar graph illustrating absorptivity of deionized water for Inventive and Comparative Examples.

FIG. 7 is a bar graph illustrating absorptivity of simulated wound fluid for Inventive and Comparative Examples.

### DETAILED DESCRIPTION

### Definitions and Terms

“Hydrophilic” is defined as having a strong affinity for or the ability to absorb water.

“Hydrophobic” is defined as lacking affinity for or the ability to absorb water.

“Non-electrically conductive” is defined as having a resistance in ohms per square inch of fabric of greater than about 10,000 ohms, preferably greater than about 100,000 ohms and most preferably greater than about 1×10⁹ ohms, when measured in accordance with AATCC Test Method 76-1978.

As utilized herein, the term “surface energy” refers to the excess energy at the surface of a material compared to the bulk of the material (e.g., the interior portions of the material) and is usually expressed in terms of milliJoules per square meter (mJ/m²). The surface energy quantifies the disruption of intermolecular bonds that occurs when a surface is created. The surface energy can be measured by several means including, for example, the Fowkes method. In this method, two reference liquids are used to first measure the dispersive component and the polar component of the material's surface energy. The surface energy of the material is then calculated from the measured dispersive and polar components. In general, a surface having a higher surface energy will exhibit a higher affinity for aqueous fluids, such as perspiration or wound exudate.

The wound care device of the present invention is generally intended to be used for treatment of various wounds including, without limitation, partial thickness burns, incisions, skin grafts, donor sites, lacerations, abrasions, Stage I-IV pressure ulcers, vascular venous stasis, and diabetic ulcers. The wound care device is generally comprised of at least two layers: a first wound contact layer comprising lyocell fiber and a second (non-wound contacting) layer formed from synthetic fibers, natural fibers, or combinations thereof. The wound contact layer may also contain additional synthetic and/or natural fibers. Thus, in one aspect of the invention, the wound contact layer of the wound care device is comprised of lyocell fiber in an amount that is in the range from about 1% by weight to about 100% by weight of the wound contact layer, or in the range from about 1% by weight to about 80% by weight, or in the range from about 1% by weight to about 60% by weight, or in the range from about 1% by weight to about 50% by weight.

In another aspect of the invention, the wound contact layer of the wound care device is comprised of a majority by weight of lyocell fiber. In this regard, the wound contact layer of the wound care device is comprised of lyocell fiber in an amount that is in the range from about 50% by weight to about 100% by weight of the wound contact layer, or in the range from about 60% by weight to about 90% by weight, or in the range from about 80% by weight to about 90% by weight.

Additional layers of material comprising the wound care device may also contain lyocell fiber. Thus, in one aspect of the invention, the second and/or subsequent layer(s)s of the wound care device may be comprised of lyocell fiber in an amount that is in the range from about 1% by weight to about 100% by weight of the second and/or subsequent layer(s), or in the range from about 1% by weight to about 80% by weight, or in the range from about 1% by weight to about 60% by weight, or in the range from about 1% by weight to about 50% by weight.

In another aspect of the invention, the second and/or subsequent layer(s) of the wound care device may be comprised of a majority by weight of lyocell fiber. In this regard, the second and/or subsequent layer(s) of the wound care device may be comprised of lyocell fiber in an amount that is in the range from about 50% by weight to about 100% by weight of the second and/or subsequent layer(s), or in the range from about 60% by weight to about 90% by weight, or in the range from about 80% by weight to about 90% by weight.

The layers of material comprising the wound care device are generally in the form of textile substrates, such as fabrics. The layers of the wound care device may be joined together through various techniques and/or joining mechanisms such as ultrasonic welding, heat or pressure lamination, the use of adhesives (such as hot melt adhesive), needle punching, hydraulic needling, sewing, stitching (such as stitch bonding), or other fiber and/or fabric layer laminating or joining processes known to those skilled in the art, or combinations thereof. Hotmelt adhesive may be applied using a lamination process. The layers may be joined together only at intermittent locations or the layers may be joined together completely.

The use of stitch bonding appears to have the effect of reducing the loft in some areas of the wound care device. Areas containing the stitch of the stitch bonding generally have reduced loft, while areas with the stitch of the stitch bonding generally have a higher loft. The presence of stitch bonding in the wound care device may lead to improved in-plane wicking of fluids away from a fluid source by providing a path for the fluids to travel. Also, stitch bonding may provide channels, or holes, which aid in the movement of fluid away from the fluid source and into the wound care device. Channels, or holes, may be created by other methods in addition to, or as an alternative to, stitch bonding. For instance, needle punching techniques may be utilized to create desirable channels for fluid movement.

In one aspect of the invention, stitch bonding provides a joining mechanism that is present through every layer of the wound care device by having a stitch (or thread) present at each layer. In another aspect of the invention, stitch bonding provides a joining mechanism whereby the stitch (or thread) penetrates every layer of the wound care device. In yet a further aspect of the invention, stitch bonding provides a joining mechanism whereby the stitch (or thread) is present at every surface of every layer of the wound care device. The presence of stitches also increases the contact area of any fluid or moisture with other layers within the wound care device. Stitch bonding may be used to join two or more layers of fabric together, and these layers of fabric may be of any fabric construction, including knitted, woven, and/or nonwoven. The wound care device may be stitch bonded with any natural or synthetic fiber type. In one embodiment, a continuous polyester fiber is employed as the stitch bonding fiber.

Synthetic fibers comprising the layers and/or joining mechanisms of the wound care device include, for example, polyester, acrylic, polyamide, polyolefin, polyaramid, polyurethane, regenerated cellulose (i.e., rayon), and blends thereof. The term “polyamide” is intended to describe any long-chain polymer having recurring amide groups (—NH—CO—) as an integral part of the polymer chain. Examples of polyamides include nylon 6; nylon 6, 6; nylon 1, 1; and nylon 6, 10. The term “polyester” is intended to describe any long-chain polymer having recurring ester groups (—C(O)—O—). Examples of polyesters include aromatic polyesters, such as polyethylene terephthalate (PET), polybutylene terephthalate (PBT), polytrimethylene terephthalate (PTT), and polytriphenylene terephthalate, and aliphatic polyesters, such as polylactic acid (PLA). “Polyolefin” includes, for example, polypropylene, polyethylene, and combinations thereof. “Polyaramid” includes, for example, poly-p-phenyleneteraphthalamid (i.e., Kevlar®), poly-m-phenyleneteraphthalamid (i.e., Nomex®), and combinations thereof. Natural fibers include, for example, wool, cotton, flax, lyocell and blends thereof.

In one aspect of the invention, lyocell fiber is included in at least the wound contact layer of the wound care device. The incorporation of lyocell into the wound care device of the present invention provides many advantages. Lyocell is generally considered an ecofriendly cellulosic fiber (100% organic). Compared to synthetic polymer fibers, cellulosic fibers possess the advantage of being biocompatible, compostable, and renewable. Natural cellulosic fibers (e.g. cotton) may be disadvantageous for use in medical applications due to the possible contamination of fibers from pesticides. Thus, synthetically made cellulosic fibers, such as viscose/modal, are generally preferred for medical applications. However, viscose/modal fiber production is based on the derivatization of cellulose using carbon disulfide (CS₂). This process is environmentally challenging as it uses not only CS₂ but also a rather high load of dissolution and spinning bath chemicals.

In general, the lyocell production process is an environment-friendly, economically viable, product-enhancing and highly flexible alternative for the manufacture of cellulose fibers. In contrast to the viscose process, no derivatization steps such as alkalization or xanthation are required to dissolve the cellulose. Instead, a melt of N-methylmorpholine-N-oxide monohydrate (NMMO) at elevated processing temperatures (approx. 100° C.) is used as a solvent. Typically, all the chemicals used in the production process are recycled. In comparison with cotton and viscose, the lyocell process therefore constitutes a significantly lower environmental burden.

Regarding its physical structure, lyocell fiber exhibits a more rounded cross section and smoother longitudinal appearance than rayon or cotton. The structure is generally more homogeneous and dense compared to viscose or cotton, both of which have core and skin. Skin tends to detrimentally prevent efficient diffusion of moisture inside the fiber.

In addition, lyocell fibers have a unique fibril structure. Fibrils (extremely small hairs) are the tiniest components which make up the fibers. Submicroscopic channels between the individual fibrils regulate absorption and release of moisture. Thus, these tiny fibrils assist in obtaining the optimum transportation of moisture. Lyocell fibers tend to absorb the moisture in a controlled and regular manner. Upon contact with the lyocell fiber, moisture is very quickly transported into the inside of the fiber. For all these reasons (and others that may not be mentioned or even fully understood), lyocell represents an ideal fiber for use in medical applications, such as in wound care devices, where active fluid management (e.g. fluid transfer, fluid absorption, fluid retention) and high levels of absorption are desired.

Thus, in one aspect of the invention, at least one layer of the wound care device contains some amount of lyocell fiber. In another aspect of the invention, at least two layers of the wound care device contain some amount of lyocell fiber. In yet a further aspect of the invention, at least three layers of the wound care device contain some amount of lyocell fiber.

The fabric layer(s) comprising the wound care device may be formed from fibers or yarns of any size, including microdenier fibers and yarns (fibers or yarns having less than one denier per filament). The fibers or yarns may have deniers that range from less than about 1 denier per filament to about 2000 denier per filament or more preferably, from less than about 1 denier per filament to about 500 denier per filament, or even more preferably, from less than about 1 denier per filament to about 300 denier per filament. Furthermore, the fabric layer(s) may be partially or wholly comprised of multi-component or bi-component fibers or yarns, which may be splittable, or which have been partially or fully split, along their length by chemical or mechanical action. The fabric layer(s) may be comprised of fibers such as staple fiber, filament fiber, spun fiber, or combinations thereof.

The fabric comprising the layers of the wound care device may be of any variety, including but not limited to, woven fabric, knitted fabric, nonwoven fabric, or combinations thereof. The fabric may optionally be colored by a variety of dyeing techniques, such as high temperature jet dyeing with disperse dyes, vat dyeing, thermosol dyeing, pad dyeing, transfer printing, screen printing, or any other technique that is common in the art for comparable textile products. If yarns or fibers are treated by the process of the current invention, they may be dyed by suitable methods prior to fabric formation, such as, for instance, by package dyeing or solution dyeing, or after fabric formation as described above, or they may be left undyed.

Other additives may be present on and/or within the target fabric and/or fiber, including antistatic agents, optical brightening compounds, opacifiers (such as titanium dioxide), nucleating agents, antioxidants, UV stabilizers, fillers, permanent press finishes, softeners, lubricants, curing accelerators, adhesives, and the like, and combinations thereof. The fabric layer(s) may also be coated or printed or otherwise aesthetically modified. Printing may be achieved, for example, by screenprinting or flexographic printing techniques.

One specific example of a knit construction that is suitable for use as at least one layer of the wound care device of the present invention is a jersey knit. A jersey knit is a circular or flat-knit fabric made with a plain stitch in which the loops intermesh in only one direction. As a result, the appearance of the face and the back of the jersey fabric is wholly different. Thus, by utilizing a jersey knit to form a fabric comprised of polyester, lyocell, and elastomeric fibers, a fabric may be constructed that is primarily polyester-containing on one side, while the opposite side of the fabric is primarily lyocell-containing. The elastomeric fiber provides some level of stretch to the fabric, which may be useful for some wounds that require, for example, a dressing to be wrapped snugly around the wound site. The elastomeric fiber, in addition to providing conformability to the wound care device, also provides some level of softness to the device. Spandex is one non-limiting example of an elastomeric fiber and may be known by the tradename Lycra®, which is available from INVISTA of Wichita, Kan.

Additionally, it may be generally known to those skilled in the art that a knit polyester fabric tends to be hydrophobic, slow to absorb liquids, and generally exhibits little or no wicking of moisture. Since polyester is hydrophobic in nature, conventional wisdom would lead one to choose a hydrophilic natural fiber, such as lyocell, or a hydrophilic synthetic fiber, such as nylon, as the wound contacting side of the wound care device. However, it was unexpectedly discovered that by placing a hydrophobic (e.g. polyester-containing) surface against the wound site and a hydrophilic (e.g. lyocell-containing) surface away from the wound site, a unique one-way, directional flow of fluid away from the wound site was achieved.

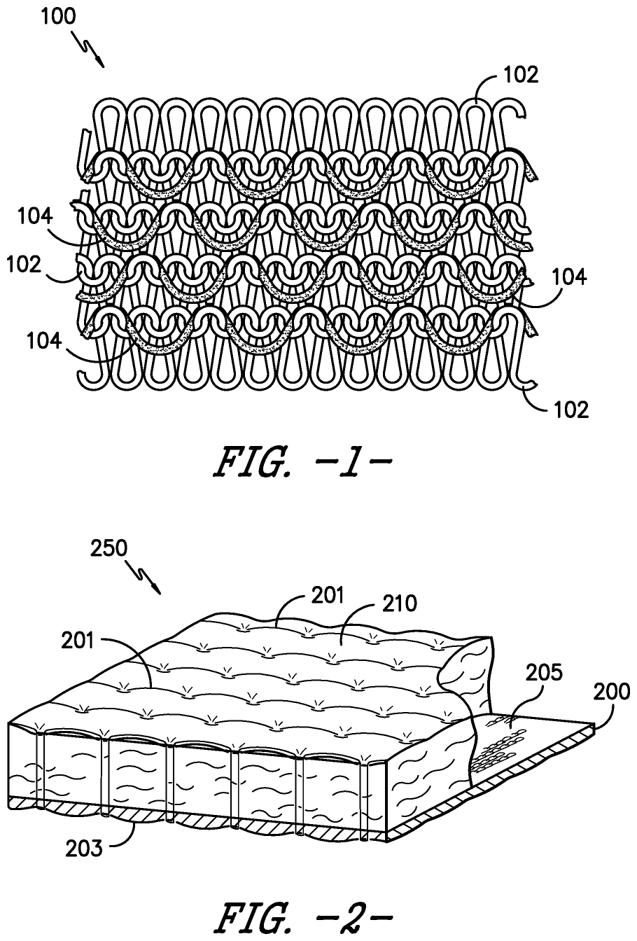

Turning now to the Figures, FIG. 1 illustrates a jersey knit construction. As shown in FIG. 1, a jersey knit construction results in knit fabric 100 in which the technical face of the fabric is predominantly one type of yarn 102, and the technical back presents a higher proportion of the effect yarn(s) 104. Thus, when the yarn 102 and the effect yarn 104 have different surface energies or one is more hydrophilic than the other, the resulting knit fabric 100 will exhibit a different surface energy on each of the two major surfaces. In a specific embodiment, knit fabric 100 (also referred to herein as the “fluid transport layer”) is comprised of yarn(s) 102 that are more hydrophilic (such as lyocell) than effect yarn(s) 104 (such as polyester). Such an embodiment of knit fabric 100 provides a layer in which the technical face of the fabric exhibits a higher surface energy than the technical back of the fabric. Thus, when utilized as the fluid transport layer of the multi-layer wound care device, the jersey knit fabric is disposed so that the technical back of the fabric forms the wound contact surface of the fluid transport layer.

This difference in surface energies between the two surfaces means that the second surface of the fluid transport layer (non-wound contact surface) exhibits a greater affinity for aqueous fluids (e.g., perspiration or wound exudates) than the first surface (wound contact surface) of the fluid transport layer. Thus, any aqueous fluids absorbed by the fluid transport layer will be transported or pumped from the first surface to the second surface of the fluid transport layer. This active transportation or pumping of the fluids ensures that excess moisture does not accumulate at the interface of fluid transport layer and a fluid exuding surface, such as the skin or an exuding wound.

When the fluid transport layer comprises first and second surfaces having different surface energies, the difference between the two surface energies can be of any suitable magnitude. In a specific embodiment, the surface energy of the second surface of the fluid transport layer can be about 101% or more of the surface energy of the first surface of the fluid transport layer. In more specific embodiments, the surface energy of the second surface can be about 102% or more, about 103% or more, or about 104% or more of the surface energy of the first surface.

While fiber types are known to be generally hydrophilic or hydrophobic in their natural or initial manufactured condition, this condition can be changed with chemical and/or physical modification to the fibers and/or textile substrates containing the fibers. For instance, polyester fiber could be made to exhibit hydrophilic properties via chemical and/or mechanical treatment. Chemical treatments that may make normally hydrophobic fibers/fabrics more hydrophilic include, for example, Visa Endurance® fabric treatment available from Milliken & Company of Spartanburg, S.C. Mechanical treatments that may make normally hydrophobic fibers/fabrics more hydrophilic include, for example, exposure to mechanical face finishing processes. Exemplary mechanical treatments include face finishing treatments like sanding, napping, calendaring, hydroentanglement with gas or liquid, and the like, and combinations thereof.

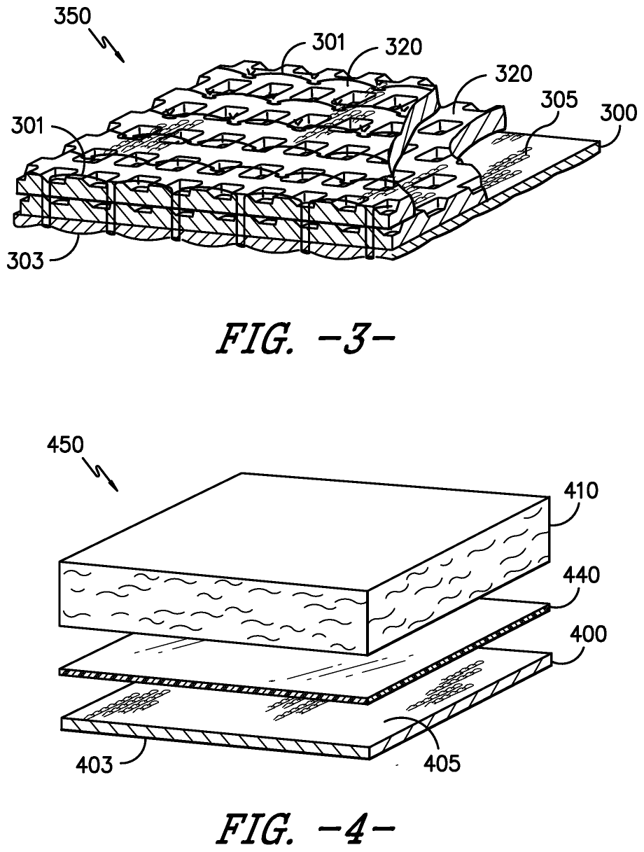

FIG. 2 illustrates a two-layer wound care device. Wound care device 250 is comprised of knit fabric 200 (which is the same as knit fabric 100 shown in FIG. 1) and nonwoven fabric 210. In one aspect of the invention, nonwoven fabric 210 is comprised of a majority by weight of lyocell fiber. Nonwoven fabric 210 is further characterized by having a wound facing surface and a non-wound facing surface. The wound facing surface is the surface of fabric 210 in closest proximity to knit fabric 200. Knit fabric 200 is the wound contact layer of wound care device 250. Nonwoven fabric 210 is joined with knit fabric 200 via stitch bonding stitch 201. Knit fabric 200 contains wound contact surface 203 and wound reservoir surface 205.

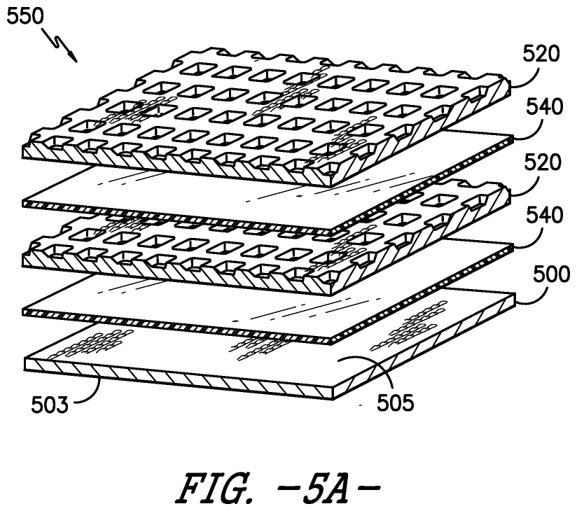

FIG. 3 illustrates a three-layer wound care device. Wound care device 350 is comprised of knit fabric 300 (which is the same as knit fabric 100 shown in FIG. 1) and two layers of waffle knit fabric 320. A waffle knit is a knit configuration that resembles a waffle, having areas in a regular pattern that are higher in elevation and lower in elevation. It is generally a porous structure. This waffle knit configuration typically provides more surface area than other standard knit configurations. In one aspect of the invention, waffle knit fabric 320 is comprised of a majority by weight of lyocell fiber. Waffle knit fabric 320 is further characterized by having a wound facing surface and a non-wound facing surface. The wound facing surface is the surface of fabric 320 in closest proximity to knit fabric 300. Knit fabric 300 is the wound contact layer of wound care device 350. Knit fabric 300 contains wound contact surface 303 and wound reservoir surface 305. The three layers comprising wound care device 350 are joined via stitch bonding stitch 301.

FIG. 4 illustrates a wound care device comprised of two layers of fabric joined with hotmelt adhesive. Wound care device 450 is comprised of knit fabric 400 (which is the same as knit fabric 100 shown in FIG. 1) and nonwoven fabric 410. In one aspect of the invention, nonwoven fabric 410 is comprised of a majority by weight of lyocell fiber. Nonwoven fabric 410 is further characterized by having a wound facing surface and a non-wound facing surface. The wound facing surface is the surface of fabric 410 in closest proximity to knit fabric 400. Knit fabric 400 is the wound contact layer of wound care device 450. Knit fabric 400 contains wound contact surface 403 and wound reservoir surface 405. Nonwoven fabric 410 is joined with knit fabric 400 via a layer of hotmelt adhesive 440. Hotmelt adhesives include, for example, polyurethane hotmelt, polyolefin hotmelt, polyamide hotmelt, co-polymers of polyurethane hotmelt, co-polymers of polyolefin hotmelt, co-polymers of polyamide hotmelt, and the like, and mixtures thereof. The hotmelt adhesive may be present as a substantially uniform layer of material across the surface of the layer(s) comprising the wound care device, as depicted in FIG. 4. Alternatively, the hotmelt adhesive may be present in a non-uniform configuration across the surface of the layer(s) comprising the wound care device.

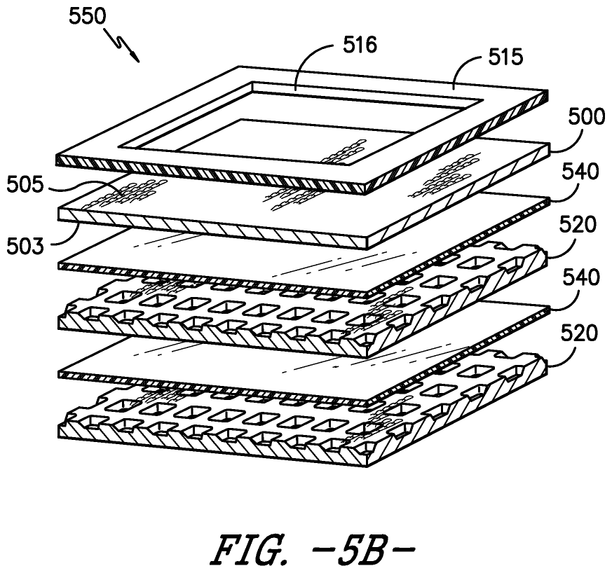

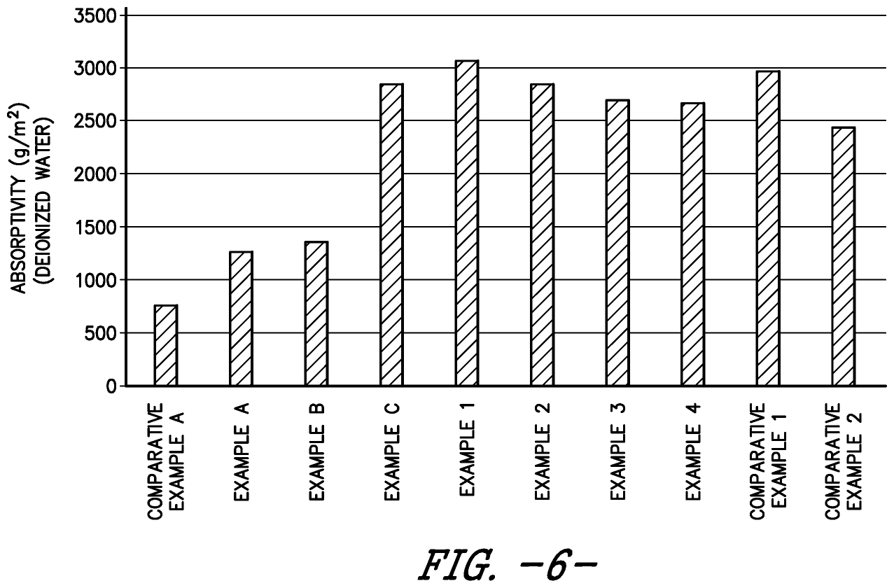

FIG. 5A illustrates a wound care device comprised of three layers of fabric joined with hotmelt adhesive. Wound care device 550 is comprised of knit fabric 500 (which is the same as knit fabric 100 shown in FIG. 1) and two layers of waffle knit fabric 520. In one aspect of the invention, waffle knit fabric 520 is comprised of a majority by weight of lyocell fiber. Waffle knit fabric 520 is further characterized by having a wound facing surface and a non-wound facing surface. The wound facing surface is the surface of fabric 520 in closest proximity to knit fabric 500. Knit fabric 500 is the wound contact layer of wound care device 550. Knit fabric 500 contains wound contact surface 503 and wound reservoir surface 505. The three layers of fabric comprising wound care device 550 are joined via hotmelt adhesive 540.

In one aspect of the invention, the layers of the wound care device may be joined using more than one type of joining mechanism. For example, in a wound care device comprised of three layers of fabric, the first and second layer may be joined via stitch bonding and the second and third layers may be joined via hotmelt adhesive. Any variations on this aspect of the invention are also contemplated to be within the scope of the present invention.

Additional layers of material may be included with the wound care device of the present invention. For example, a fluid retentive layer may be attached to the fabric layer. The fluid retentive layer may be attached using hot melt adhesive. Also, an occlusive (non-perforated) or perforated film layer may be attached to the wound care device. The film layer may be attached using hot melt adhesive. An adhesive layer comprised of at least one adhesive material may be added to the wound care device. The adhesive layer may be provided to aid in adhering the wound care device to the skin and/or wound site. Suitable adhesive materials are selected from the group consisting of natural rubber-based adhesive materials, synthetic rubber-based adhesive materials, hydrocolloid materials, acrylate and/or acrylic materials, polyurethane gel materials, polydimethylsiloxane materials, and the like, and mixtures thereof. In addition, one or more of the following types of adhesive materials may be suitable for use as the adhesive layer of the wound care device of the present invention:

TABLE A
Types of Adhesive Materials

Ultraviolet and/or Visible Acrylics and/or Acrylates
Indigo Visible Acrylics and/or Acrylates
Flashcure Cyanoacrylates
Silicones
Cyanoacrylates
Polyurethane Gel
Polyurethane
Synthetic Rubber
Surface Insensitive
Low Odor and/or Low Bloom
Toughened and/or Flexible
General Purpose
Primers and/or Accelerators
One-Part Heat Cure Epoxies
Two-Part Room Temperature Cure Epoxies and/or Urethanes
Thermally Conductive Compound
Thermally Conductive Gel

In one aspect of the invention, the adhesive layer may be included with the wound care device as a layer of material having substantially the same dimensions as the wound care device. In another aspect of the invention, the adhesive layer may have an opening (or window) in the approximate center of the adhesive layer. FIG. 5B illustrates this feature. Adhesive layer 515 is present as a border adhesive for wound care device 550. FIG. 5B also illustrates opening 516 in the approximate center of adhesive layer 515.

Further, a release liner may be included as part of the packaging of the wound care device. The release liner is intended to be removed prior to use of the wound care device. The release liner may be comprised of material selected from the group consisting of polycarbonate, polypropylene, polyethylene, coated paper, and the like, and combinations thereof. The release liner may be printed.

The fluid retentive layer, if included with the wound care device, may be selected from the group consisting of foams, textile materials (e.g. woven, knit, and nonwoven textile materials), alginates, superabsorbent polymers, gels (e.g., hydrogels), and combinations or mixtures thereof. The fluid retentive layer can also comprise a combination of two or more discrete layers, which layers can comprise any of the absorptive materials listed above. In a specific embodiment, the fluid retentive layer can be a foam, such as an open cell, non-reticulated polymer foam. Such foams can be made from any suitable material including, but not limited to, polyurethane polymers. In one aspect, a polyurethane polymer used in making such a foam can be a polyester-based polyurethane polymer (i.e., a polyurethane polymer made from a reaction mixture containing a polyester polyol).

The fluid retentive layer of the wound care device may exhibit any suitable absorptive capacity. For example, the fluid retentive layer may exhibit a fluid absorption of about 100 wt % or more based on the weight of the fluid retentive layer. In a specific embodiment, the fluid retentive layer may exhibit a fluid absorption of about 200 wt % or more, about 300 wt % or more, about 400 wt % or more, about 500 wt % or more, about 600 wt % or more, about 700 wt % or more, about 800 wt % or more, about 900 wt % or more, or about 1000 wt % or more based on the weight of the fluid retentive layer. The absorptive capacity of the fluid retentive layer may be measured by any suitable means. For example, the absorptive capacity of the fluid retentive layer may be measured by immersing a known weight of the fluid retentive layer in phosphate-buffered saline containing 0.9 wt % sodium chloride at 37° C. for 30 minutes.

Any of the optional layers described herein may or may not be substantially coextensive with the fabric layer(s) comprising the wound care device. One or more layers of the wound care device may be printed with a product logo or other product identification information.

The wound care device of the present invention may be of any thickness, depending on the construction of the fabric and the number of layers included therein. In one aspect, the thickness of the wound care device may be in the range from about 25 to about 60 mils, or in the range from about 35 to about 50 mils, or even in the range from about 38 to about 45 mils. Thickness measurements may be increased when the wound care device also includes an antimicrobial finish on one or more surfaces of the wound care device.

An antimicrobial treatment may be applied to one or more layers comprising the wound care device. In one aspect of the invention, the antimicrobial treatment is added to one or more fabric layers of the wound care device. In a further aspect of the invention, the antimicrobial treatment is added to the wound contact surface of the wound care device.

The particular antimicrobial treatment which may be applied to the wound care device of the present invention comprises at least one silver-containing compound selected from the group consisting of silver ion exchange materials (e.g. silver zirconium phosphates, silver calcium phosphates and silver zeolites), silver particles (e.g. silver metal, nanosilver, colloidal silver), silver salts (e.g. AgCl, Ag₂CO₃), silver glass, and mixtures thereof. One preferred silver-containing compound is an antimicrobial silver sodium hydrogen zirconium phosphate available from Milliken & Company of Spartanburg, S.C., sold under the tradename AlphaSan®. Other potentially preferred silver-containing antimicrobials suitable for use herein—including silver zeolites, such as a silver ion-loaded zeolite available from Sinanen Co., Ltd. of Tokyo, Japan under the tradename Zeomic®, and silver glass, such as those available from Ishizuka Glass Co., Ltd. of Japan under the tradename lonpure®—may be utilized either in addition to, or as a substitute for, the preferred species listed above. Other silver-containing materials may also be used. Various combinations of these silver-containing materials may be made if adjustments to the silver release rate over time are desired.

Generally, the silver-containing antimicrobial compound is added in an amount from about 0.01% to about 60% by total weight of the particular finish composition; more preferably, from about 0.05% to about 40%; and most preferably, from about 0.1% to about 30%. The antimicrobial finish itself, including any desired binders, wetting agents, odor absorbing agents, leveling agents, adherents, thickeners, and the like, is added to the wound care device in an amount of at least about 0.01% of the total wound care device weight.

A binder material has been found useful in preventing the antimicrobial from flaking onto the wound. Preferably, this component is a polyurethane-based binding agent, although a wide variety of cationic, anionic, and non-ionic binders may also be used, either alone or in combination. Preferably, the binding agent is biocompatible such that it does not cause negative reactions in the wound. In essence, such binders provide durability by adhering the antimicrobial to the target substrate, such as fibers or fabrics, without negatively affecting the release of silver to the wound.

Total add-on levels of silver to the target substrate may be 20 ppm or higher. More preferably, total add-on levels of silver may be 200 ppm or higher. Although an upper boundary limit of silver add-on levels to the target substrate has not been determined, consideration of the manufacturing economics and the potential to irritate a sensitive wound site suggests avoiding excessive silver levels.

An additional advantageous feature of the silver-containing wound care device of the present invention is its ability to substantially maintain its original color, despite the presence of effective amounts of a silver-based antimicrobial agent. The elimination of color normally associated with the inclusion of silver-based antimicrobials is highly beneficial and desirable. The wound care devices (preferably, white-colored) allow users thereof and their health care providers to monitor the exudates from the wound. Further, the present wound care devices exhibit long-term color stability (that is, their color does not change significantly over time while in production, transit, or storage). Finally, because the present wound care device is not discolored by the addition of the silver-containing antimicrobial agent, a variety of substrate colors may be utilized or the finished wound care devices may be dyed or colored to any desired shade or hue with any type of colorant, such as, for example, pigments, dyes, tints, and the like. Thus, one or more layers of the wound care device may contain a coloring agent. The coloring agent is selected from the group consisting of pigments, dyes, tints, and the like, and combinations thereof.

Silver-containing compounds (such as AlphaSan®, Zeomic®, or lonpure®) may be admixed in an aqueous dispersion with a binder to form a bath into which the target substrate is immersed. The target substrate includes any of the layers comprising the wound care device of the present invention. Other similar types of compounds that provide silver may also be utilized.

When specific polyurethane-based binder materials are utilized, the antimicrobial characteristics of the treated substrate are effective with regard to the amount of surface available silver that is released to kill bacteria, without altering the color of the treated substrate (that is, while substantially maintaining its original appearance). While it currently appears that the use of polyurethane-based binder resins are preferred due to their allowance of silver release and bio-neutral properties, in practice essentially any effective cationic, anionic, or non-ionic binder resin that is not toxic to the wound may be used.

An acceptable method of providing a durable antimicrobial silver-treated fabric surface is the application of a silver-containing compound and polyurethane-based binder resin from a bath mixture. This mixture of antimicrobial compound and binder resin may be applied through any technique as is known in the art, including spraying, dipping, padding, foaming, printing, and the like. By using one or more of these application techniques, a fabric may be treated with the antimicrobial compound and binder resin on only one side of the fabric (e.g. the wound contact surface of a wound care device), or it may be treated on both sides of the fabric.

The wound care device of the present invention may be cut into any geometric shape or size depending upon its end-use application. The wound care device may be cut using a computer controlled cutting device such as a Gerber machine. It may also be cut using a mechanical dye cutter, hot knife, straight blade, or rotary blade. The wound care device may be cut into any size, such as, for example, a square, rectangle, triangle, circle and the like. The length of the wound care device may be 1″, 2″, 3″, 4″, 5″, 6″, 7″, and the like and longer. The width may be 1″, 2″, 3″, 4″, 5″, 6″, 7″, and the like and longer. The wound care device may be comprised of any combination of length and width. In one aspect, the wound care device may be 2″ by 2″, 2″ by 3″, 3″ by 3″, 4″ by 2″, 4″ by 3″, 4″ by 4″, or 4″ by 5″ in size. The wound care device may also be of any variety of whimsical shapes, such as, dog bone shape, heart shape, smiley face, or any other shape that is desired. The wound care device may also be sterilized prior to use via a variety of heat, chemical and/or radiation techniques. In one aspect, sterilization may be accomplished via gamma radiation.

The following examples further illustrate the present multi-layer wound care device having absorption and fluid transfer properties, but are not to be construed as limiting the invention as defined in the claims appended hereto. All parts and percents given in these examples are by weight unless otherwise indicated.

### EXAMPLES

### Sample Creation and Evaluation

### Substrate Descriptions:

Fabric A was a jersey knit (circular knit), multi-polymer fabric sold by Milliken & Company. Fabric A was a single layer of fabric comprised of approximately 85% ring spun lyocell yarn, 10% continuous filament polyester yarn, and 5% continuous filament spandex yarn. Fibers used for lyocell yarn was commercially available as Tencel™ fiber (from Lenzing). Lyocell yarns were package dyed with a reactive dye. The lyocell yarn was comprised of 22.8/1 cc (cotton count) and the undyed fibers were 1.4 dtex, 51 mm. Tenacity of these fibers were >32 cN/tex and elongation was >10%. The polyester yarn was comprised of single ply 70 denier/34 filament count fiber that was exposed to a texturing process prior to knitting. The spandex yarn was comprised of 55 denier/3 filament count fiber. The fabric was knitted in such a manner as to give a distinct lyocell side and a distinct polyester side.

Fabric B was a circular knit waffle fabric comprised of a majority by weight of lyocell fiber. Fabric B was a single layer of fabric comprised of 77% lyocell fiber and 23% polyester fiber. Fibers used for lyocell yarn was commercially available as Tencel™ fiber (from Lenzing). Lyocell yarns were package dyed with a reactive dye. The lyocell yarn was comprised of 22.8/1 cc (cotton count) and the undyed fibers were 1.4 dtex, 51 mm. Tenacity of these fibers were >32 cN/tex and elongation was >10%. The polyester yarn was comprised of single ply 70 denier/34 filament count fiber that was exposed to a texturing process prior to knitting. Fabric B was a double-knit fabric with tuck stitches used to create a waffle type appearance. One surface of Fabric B has a greater amount polyester, and the other surface has a greater amount of lyocell. Fabric B was designed to have higher absorbency than Fabric A.

Fabric C was a single layer of nonwoven fabric comprised of 50% lyocell and 50% polyester fiber. Lyocell fibers were 1.4 dtex/51 mm and polyester fibers were 7 dtex/51 mm. The nonwoven was made by a needle punch process.

The hotmelt adhesive used to laminate the layers of fabric together was a co-polyamide Vilmed® M 1591 available from Freudenberg Performance Materials. Weight of the hot melt adhesive was 20 g/m2 and melting range was 110-130° C.

The stitch bonding thread was bonded polyester—size 92/Tex 90.

The following samples were tested for absorptivity:

Example A:

Layer 1: Fabric A

Example B:

Layer 1: Fabric B

Example C:

Layer 1: Fabric C

Example 1:

Layer 1: Fabric A

Layer 2: Hotmelt adhesive

Layer 3: Fabric B, 1^{st} instance

Layer 4: Hotmelt adhesive

Layer 5: Fabric B, 2^{nd} instance

Fabric A was laminated with co-polyamide hotmelt adhesive to Fabric B (1^{st} instance) in such a configuration that the primarily lyocell-containing surface of Fabric A (the non-wound contact surface) was facing the polyester rich surface of Fabric B (1^{st} instance). Then, this three-layer composite material was laminated with co-polyamide hotmelt adhesive to Fabric B (2^{nd} instance) in such a configuration that the lyocell rich surface of Fabric B (1^{st} instance) was facing the polyester rich surface of Fabric B (2^{nd} instance). Example 1 is shown in FIG. 5A.

During the lamination process, the desired layers were assembled and then laminated using a belt laminator. All laminator heating zones were set to 140° C.

Dwell time for lamination was set to 1 minute. Laminator tunnel height was 5 mm. Layers were sent through the laminator between two sheets of liner paper.

Example 2:

Layer 1: Fabric A

Layer 2: Fabric B, 1^{st} instance

Layer 3: Fabric B, 2^{nd} instance

Layers 1-3 were stitch bonded together using the polyester stitch bonding thread described above. Fabric A was stitch bonded to both layers of Fabric B in such a configuration that the primarily lyocell-containing surface of Fabric A (the non-wound contact surface) was facing the polyester rich surface of Fabric B (1^{st} instance), which was facing the polyester rich surface of Fabric B (2^{nd} instance). Example 2 is shown in FIG. 3.

Example 3:

Layer 1: Fabric A

Layer 2: Hotmelt adhesive

Layer 3: Fabric C

Layers 1 and 3 were bonded together with co-polyamide hotmelt

Fabric A was laminated with co-polyamide hotmelt adhesive to Fabric C in such a configuration that the primarily lyocell-containing surface of Fabric A (the non-wound contact surface) was facing Fabric C. Example 3 was shown in FIG. 4.

During the lamination process, the desired layers were assembled and then laminated using a belt laminator. All laminator heating zones were set to 140° C. Dwell time for lamination was set to 1 minute. Laminator tunnel height was 5 mm. Layers were sent through the laminator between two sheets of liner paper.

Example 4:

Layer 1: Fabric A

Layer 2: Fabric C

Layers 1 and 2 were stitch bonded together using the polyester stitch bonding thread described above. Fabric A was stitch bonded to Fabric C in such a configuration that the primarily lyocell-containing surface of Fabric A (the non-wound contact surface) was facing Fabric C. Example 4 is shown in FIG. 2.

Comparative Example b 1:

Comparative Example 1 was Drawtex®, a nonwoven fabric comprised of a blend of 72% viscose fiber and 28% polyester fiber commercially available from Beier Drawtex Heathcare of Fort Worth, Tex.

### Comparative Example 2:

Comparative Example 2 was Aquacel®, a 100% carboxymethylcellulose (“CMC”) nonwoven fabric stitch bonded with cellulosic yarn commercially available from Convatec of Bridgewater, N.J.

### Absorptivity Evaluation:

Each of the Examples and Comparative Examples was tested for absorptivity according to the following test method:

Absorptivity Test Method: For each sample tested, a 2″ diameter circular disk was cut from the sample and submerged into a glass jar containing fluid that was: (a) deionized water or (b) simulated wound fluid (“SWF”). Simulated wound fluid is a solution of deionized water containing 142 mM of sodium chloride and 2.5 mM of calcium chloride.

Each sample was equilibrated for 30 minutes at 37° C. under the submerged condition. Free swell absorptivity of the fluid was then calculated by subtracting the weight of the dry disk (in grams) from the weight of disk with absorbed fluid (in grams). This weight was divided by the area of the disk (in square meters) and used to calculate the free well absorptivity of the sample.

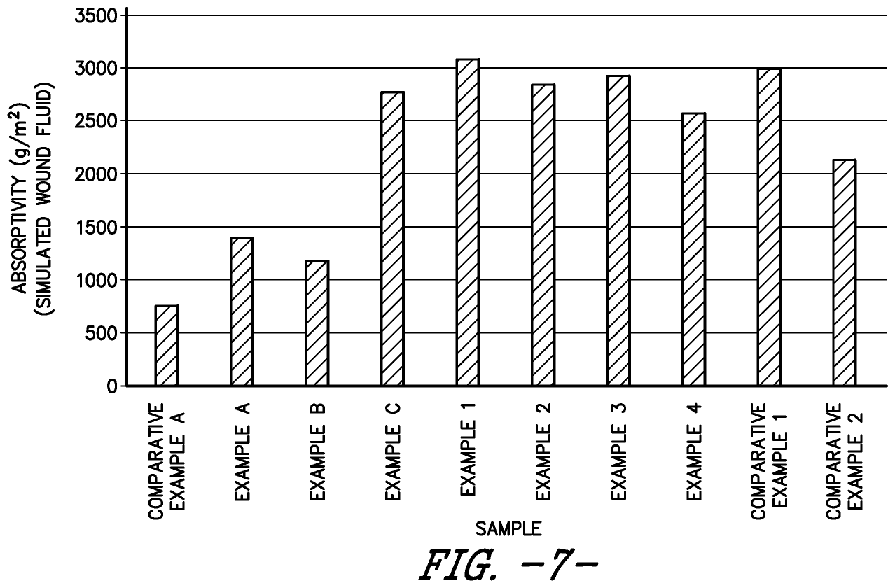

Test results are shown in Table 1 and FIG. 6 and FIG. 7.

TABLE 1
Free Swell Absorbency of Inventive And Comparative Examples

| | Absorptivity (g/m²) | Absorptivity (g/m²) |
|---|---|---|
| Sample | (Deionized Water) | (SWF) |
| Comparative Example A | 768 | 762 |
| Example A | 1275 | 1406 |
| Example B | 1368 | 1193 |
| Example C | 2852 | 2788 |
| Example 1 | 3071 | 3102 |
| Example 2 | 2849 | 2867 |
| Example 3 | 2695 | 2951 |
| Example 4 | 2663 | 2600 |
| Comparative Example 1 | 2969 | 3021 |
| Comparative Example 2 | 2432 | 2162 |

Further evaluation of select samples in deionized water was made using optical microscopy to determine absorption features at the fiber level of Comparative Example 1, Comparative Example 2 and Example A.

Optical microscopy images showed that the viscose/polyester fibers of Comparative Example 1 exhibited no significant change in fiber diameter, indicating that little to no moisture absorption occurred. The CMC fibers of Comparative Example 2 swelled so much that the fibers actually burst, indicating that absorption was limited to the point prior to burst and that the fiber had little to no structural integrity to maintain moisture. The lyocell fiber of Example A exhibited (on average) an 18% increase in fiber diameter, indicating that the fiber was capable of absorbing and holding a significant amount of moisture.

### Staining Evaluation:

To further illustrate the superior absorption property of lyocell fiber, staining experiments were performed using an aqueous solution of methylene blue. Three different jersey knit fabrics were tested—100% polyester, 100% viscose, and 100% lyocell.

Each fabric was dipped into the methylene blue solution for 2 minutes. The fabric was then removed and rinsed with deionized water. Based on visible observation, darker staining for the lyocell fabric was clearly achieved compared to viscose. The polyester fabric exhibited a very light amount of blue staining. This visible observation confirmed deeper penetration of moisture (along with the dye) in lyocell fibers.

Optical microscopy was employed for further evaluation of individual fibers containing the aqueous methylene blue solution. Looking at cross sections of each stained fiber type, optical microscopy images also confirmed darker and more complete absorption of moisture by the lyocell fiber.

### Periwound Protection Evaluation:

The purpose of this test was to measure the amount of moisture or liquid that is transferred from the wound care device to healthy periwound skin. Simulated wound fluid (“SWF”) was used as the liquid. The apparatus was a syringe pump with 1/32″ internal diameter tubing attached to a small hole in the center of a petri dish. A pre-weighed, 2″ diameter piece of two-layer gauze with a 10 mm hole in the center was placed on the petri dish with the tubing hole centered within the hole in the gauze. The gauze simulated the periwound skin. The syringe pump is used to pump SWF with blue colored dye to simulated wound beds. Simulated wound beds are created using petri dishes with tubing followed by two-layer gauze disks with a centered circular cut-out. The wound care device was placed on this gauze disk, followed by absorbent gauze and a weighted disk. SWF was pumped through the tubing into the periwound setup at a set rate of 0.2 mL/h for 24 hours.

SWF absorbed by the gauze represented the moisture that remained in the periwound instead of being taken up into the wound care device. Percent periwound pick up was calculated from the amount of SWF absorbed by the simulated periwound divided by its initial weight. Lower % periwound pick up is ideal for wound healing and lowers the probability of periwound maceration. Test results are provided in Table 2.

TABLE 2
Percent Periwound Pick Up

| Sample | % Periwound pick up |
|---|---|
| Example 1 | 29% |
| Comparative Example 1 | 139% |
| Comparative Example 2 | 130% |

The data in Table 2 demonstrates that lower % periwound pick up is achieved with the wound care device of Example 1 compared to Comparative Example 1 and 2.

Tensile Strength (Adapted from ASTM D5034-09(2017)):

Testing was carried out on a universal force testing machine (i.e., Sintech tensile test machine). Test strips of each sample were prepared at 2 inches wide, 4 inches long. Clamps were set at 2 inches apart, and the machine speed was set at 300 mm/min. Samples were then tested for break strength. To prepare wet samples, the strips were prepared in the clamps as with the dry samples. Approximately 15 mL of DI water were added to the samples via syringe, to saturation. The water was added to the sample between the clamps. Once the sample between the clamps was saturated, the breaking force test was carried out in the same manner as with the dry samples. Tensile strength was measured and recorded as pounds of force (“lbf”). Strength Retention is calculated by dividing the wet value by the dry value. Test results are provided in Table 3.

TABLE 3
Tensile Strength

| | Tensile | Tensile | |
|---|---|---|---|
| | Strength - Dry | Strength - Wet | Strength |
| Sample | (lbf) | (lbf) | Retention |
| Example 1 | 175 | 164 | 94% |
| Comparative Example 1 | 181 | 99 | 55% |
| Comparative Example 2 | 43 | 5 | 12% |

As presented in Table 3, Example 1 retains greater than 90% of its strength upon absorption of moisture.

Additional test procedures are described herein below. Many of these tests were conducted in commonly owned U.S. Pat. Nos. 7,842,306; 8,021,685, and 8,394,403, all of which are incorporated by reference herein.

### TEST 1: Drop Disappearance Test

The purpose of this test is to measure the amount of time it takes for a single drop of fluid to be absorbed into the substrate. The fluid used was simulated wound fluid. Simulated wound fluid is a solution of deionized water containing 142 mM of sodium chloride and 2.5 mM of calcium chloride. The simulated wound fluid is isotonic to human blood. The simulated wound fluid was contained within a 2 mL syringe. Two millimeters of fluid were dispensed by hand onto the approximate center of the substrate. The time it took for the drop to disappear (to be absorbed into the substrate) was recorded. The test was stopped after 600 seconds was reached.

### TEST 2: Vertical Lea Model Test

The purpose of this test is to measure the amount of fluid that is absorbed by the wound care device over a period of time in a vertical orientation prior to failure. The fluid used was simulated wound fluid. Failure is defined as the point in time when the wound care device either (a) started to peel from the nylon surface of the leg model or completely fell off the leg model, or (b) started to leak simulated wound fluid from the edges and/or borders of the wound care device. Samples were run at 24 mL/hour until failure.

### TEST 3: Vertical Wicking Test

The purpose of this test is to measure the amount of fluid that is absorbed by the wound care device over a certain period of time in a vertical orientation. The fluid used was simulated wound fluid. Each sample was tested in triplicate. The average and standard deviation was calculated.

### TEST 4: Peel Strength Test

The purpose of this test is to measure the amount of force it takes to remove the wound care device from the surface of stainless steel. Each sample was applied to the surface according to the product directions. Removal of the sample was done by a testing machine with a load weighing system. The force required to remove each sample was recorded in grams of force (gf).

### TEST 5: Antimicrobial Efficacy

Antimicrobial efficacy against both Gram-positive (e.g. Staphylococcus aureus ATCC #6538) and Gram-negative (e.g. Klebsiella pneumoniae ATCC #4352) bacteria was measured for inventive and comparative wound care devices. The quantitative reduction of bacteria after exposure to the samples versus the control was assessed using a modified version of AATCC Method 100.

Portions of each wound dressing sample (non-sterile 15 mm diameter disks) were placed into 24-well microplates. With all samples, the dressings were placed with the side down that normally contacts the wound. Overnight cultures of the test microbes were suspended in 5% nutrient broth in saline ca. 10E6 cells/ml. At time 0, each sample was pre-soaked in sterile saline via immersion. The wells of the 24 well plate were inoculated with bacteria ( 0.1 ml of ca. 10E6 cells/ml) and then the sample was placed contact side down in the inoculum. The 24 well plates were then incubated at 37° C. After incubation for 24 hours, the samples were removed and placed into 50 ml centrifuge tubes filled with 5 ml of a “wash solution” (Tryptic Soy Broth+0.7% Tween 80+0.1% cysteine (to inactivate residual silver)). After vortexing to remove attached cells, the number of viable cells in the solution was quantified using a microtiter plate-based “Most-Probable Number” assay. The recipe for full-strength Nutrient Broth indicated in this method is 5 g/l peptone and 3 g/l beef extract. Duplicate samples were tested against Staphylococcus aureus ATCC#6538 and Klebsiella pneumoniae ATCC#4352.

### TEST 6: Fluid Transport Test

The purpose of this test is to measure the amount of fluid that is transported from the wound contact side of the wound care device (Side A) to the non-wound contact side of the device (Side B). The test also attempts to measure the amount of fluid pushed back to the wound contact side of the device (Side A).

Simulated wound fluid (“SWF”) was prepared by adding 16.60 g NaCl and 0.56 g CaCl₂ to a 2 L volumetric flask. The flask was then filled to volume (2000 mL total) with deionized water. The flask was then capped and shaken until all of the salts were completely dissolved. The simulated wound fluid is comprised of 0.142M (142 mM) NaCl (aq) and 0.0025M ( 2.5 mM) CaCl₂ (aq).

A test sample of a wound care device (5 cm in diameter) was placed onto a polypropylene disc (5 cm in diameter). Twenty drops of simulated wound fluid was added to Side A of the test sample using a dropper. The test sample was allowed to rest in a horizontal position for 2 minutes. The test sample was then sandwiched in a vertical position between two discs of filter paper (Whitman filter paper 3, diameter=110 mm) using a clamp—Filter Paper A contacted Side A of the test sample and Filter Paper B contacted Side B of the test sample. The test sample was held in this position for 5 seconds. It was determined that the clamp exerts a pressure of 340 mm Hg.

Filter papers A and B had been weighed prior to the test. They were then weighed after the test and difference in weight was determined. This weight difference provides a calculation of the amount of SWF transferred from the wound care device to Filter Paper A and/or B.

The SWF was added to the polyester side (“Side A”) of the wound care device of the present invention. SWF was added to the wound contact side of competitive dressings, as directed by the product brochures.

The values are provided as “percent weight change.” The percent weight change represents the weight of the fluid absorbed relative to the dry weight of the filter paper. It is calculated by subtracting the weight of the dry filter paper (grams) from the weight of the wet filter paper (grams) and dividing this difference by the weight of the dry filter paper. This value is then multiplied by 100.

### TEST 7: Tensile Strength

Tensile strength (grab) of various wound care devices was determined using ASTM D5034. The purpose of this test is to determine structural integrity of wet and dry wound care devices. The devices were wetted by dipping them in simulated wound fluid (same formulation as described previously). Measurements are shown in pounds of force (lbf). Higher values indicate that more force was needed to tear the sample.

### TEST 8: Zone of Inhibition Test

Zone of Inhibition testing may be conducted to determine the antimicrobial activity of various wound care devices against several microbes using a modified version of the Kirby-Bauer Susceptibility Test. A brief description of the test method is included below. A full description of the test method may be found in the following document: National Committee for Clinical Laboratory Studies (NCCLS) M2-A8: Performance Standards for Antimicrobial Disk Susceptibility Tests; Approved Standard—Eighth Edition;2003.

Several Gram-positive and Gram-negative bacteria as well as fungi (yeast) may be chosen to illustrate the antimicrobial efficacy of the inventive wound care device. Gram-positive bacteria include, for example and without limitation, Staphylococcus aureus, Clostridium perfringens, Enterococcus faecium and Bacillus cereus. Gram-negative bacteria include, for example and without limitation, Klebsiella pneumoniae, Escherichia coli, Acinetobacter baumannii, Enterobacter cloacae, Proteus mirabilis, and Pseudomonas aeruginosa. Fungi, such as yeast, include for example, Candida albicans and Saccharomyces cerevisiae.

An overnight culture of the test microbe was diluted into saline (0.85% NaCl) to a concentration of 10⁶ cells/ml. Petri dishes containing Diagnostic Sensitivity Test (DST) Agar were inoculated with 0.25 ml of the cell suspension and incubated for 1 hour. A sample (15 mm diameter circle) of each wound care device was then placed at the center of the agar plate. The agar plate was incubated for 24 hours at 37° C. After measuring the extent of the zones (in mm), the samples were transferred to a fresh DST plate inoculated with the same microbe. The process was repeated for three days (total).

### TEST 9: Total AlphaSan® Content

### Total ALPHASAN® Content Test

The amount of AlphaSan® antimicrobial incorporated into or onto an article can be determined by measurement of elements unique to the antimicrobial compound. For AlphaSan® antimicrobial, the two elements of highest abundance are silver or zirconium. Because zirconium is more abundant in the AlphaSan® antimicrobial product and is easier to measure, it is preferable to use zirconium as the signature element for determining the level of AlphaSan® antimicrobial in an article. The amount of AlphaSan® antimicrobial incorporated into or onto the wound care device was determined using the following ashing technique.

A sample of fabric (weighing approximately 1 gram but with weight measured to four significant digits) was placed in a clean, dry ceramic crucible which had been weighed. The crucible containing the fabric sample was placed in a muffle furnace whose temperature ramped up at 3° C./minute to 750° C. The temperature was then held at 750° C. for four hours. The system was then cooled and the crucible transferred to a desiccator in which it was allowed to reach an equilibrium temperature. The crucible was then weighed. This provides the percent solids of inorganic constituents.

The fabric sample was then ground in the ceramic crucible to obtain a uniform sample. Approximately 0.05 g weight (again measured to four significant digits) was then taken from the ceramic crucible and placed in a platinum crucible. Four milliliters of 50% HNO₃, followed by 15-20 drops of 48% HF, were added to the crucible. The crucible was heated over a hot plate until the sample completely dissolved. The sample solution was then transferred to a 100 mL volumetric flask.

The crucible was then rinsed with 5% HNO₃, with the rinse solution being added to the flask. The solution was diluted to the 100 mL mark with 5% HNO₃. The dilute solution was transferred to a polyethylene storage container. Analysis for the desired active ingredient (in this case, zirconium) was performed using an Inductively Coupled Plasma Optical Emission Spectrometer device (e.g., a Perkin Elmer Optima 4300DV). Calculations are apparent to one skilled in the art. The amount of AlphaSan® RC2000 present on the wound care device is provided as a weight percent based on the weight of the fabric.

### TEST 10: Conductivity/Resistivity Test

The purpose of this test is to determine the conductivity and resistivity (R) of the inventive wound care device. The test was performed according to AATCC Test Method 76.

### TEST 11: Thickness Test

The purpose of this test was to measure the thickness of the inventive wound care device. The test was performed according to ASTM D 1777-96.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the subject matter of this application (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the subject matter of the application and does not pose a limitation on the scope of the subject matter unless otherwise claimed. No language in the specification should be construed as indicating any non-claimed element as essential to the practice of the subject matter described herein.

Preferred embodiments of the subject matter of this application are described herein, including the best mode known to the inventors for carrying out the claimed subject matter. Variations of those preferred embodiments may become apparent to those of ordinary skill in the art upon reading the foregoing description. The inventors expect skilled artisans to employ such variations as appropriate, and the inventors intend for the subject matter described herein to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

We claim:

1. A wound care device comprising:
a first layer of fabric having a wound contact surface and a wound fluid reservoir surface, wherein the first layer of fabric contains lyocell fibers;
a second layer of fabric, wherein the second layer of fabric has a wound facing surface and a non-wound facing surface; and
at least one joining mechanism;
wherein the at least one joining mechanism is in direct physical contact with the first layer of fabric and the second layer of fabric, and
wherein the at least one joining mechanism is present on the wound contact surface of the first layer of fabric and on the non-wound facing surface of the second layer of fabric; and
wherein the wound care device transports wound fluid uni-directionally from the wound contact surface to the wound fluid reservoir surface upon exposure to a wound.

2. The wound care device of claim 1, wherein the first layer of fabric is knit fabric.

3. The wound care device of claim 2, wherein the knit fabric is circular knit fabric.

4. The wound card device of claim 3, wherein the circular knit fabric is a jersey knit fabric.

5. The wound care device of claim 1, wherein the first layer of fabric is comprised primarily of hydrophobic fibers and hydrophilic fibers.

6. The wound care device of claim 5, wherein the hydrophobic fibers are polyester fibers.

7. The wound care device of claim 5, wherein the hydrophilic fibers are lyocell fibers.

8. The wound care device of claim 5, wherein the first layer of fabric further comprises an elastomeric fiber.

9. The wound care device of claim 1, wherein the second layer of fabric is selected from the group consisting of nonwoven fabric and knit fabric.

10. The wound care device of claim 1, wherein the second layer of fabric contains lyocell fibers.

11. The wound care device of claim 1, wherein the second layer of fabric has a fabric construction different from the first layer of fabric.

12. The wound care device of claim 1, wherein the at least one joining mechanism is stitch bonding.

13. The wound care device of claim 1, wherein the first layer of fabric is coated with a composition comprising at least one silver-containing compound.

14. The wound care device of claim 13, wherein the at least one silver-containing compound is selected from the group consisting of silver ion exchange materials, silver particles, silver salts, silver glass, and mixtures thereof.

15. The wound care device of claim 14, wherein the silver ion exchange material is selected from the group consisting of silver zirconium phosphate, silver calcium phosphate, silver zeolite, and mixtures thereof.

16. The wound care device of claim 15, wherein the silver ion exchange material is silver zirconium phosphate.

17. The wound care device of claim 13, wherein the composition further comprises a binding agent selected from the group consisting of polyurethane binders, acrylic binders, and mixtures thereof.

18. The wound care device of claim 17, wherein the binding agent is a polyurethane-based material.

19. The wound care device of claim 13, wherein the device is non-electrically conductive.

20. The wound care device of claim 1, wherein the wound contact surface and the fluid reservoir surface are coated with a composition comprising at least one silver-containing compound.

21. The wound care device of claim 20, wherein the device exhibits antimicrobial efficacy.

22. The wound care device of claim 1, wherein the device further includes a third layer of fabric.

23. A wound care device comprising:
a first layer of fabric having a wound contact surface and a wound fluid reservoir surface, wherein the first layer of fabric contains lyocell fibers;
a second layer of fabric, wherein the second layer of fabric contains lyocell fibers; and
at least one joining mechanism,
wherein the at least one joining mechanism joins the first layer of fabric to the second layer of fabric; and
wherein the wound care device transports wound fluid uni-directionally from the wound contact surface to the wound fluid reservoir surface upon exposure to a wound.

24. A method for managing moisture at a wound site comprising the steps of:
(a) providing a wound care device comprising:
(i) a first layer of fabric having a wound contact surface and a wound fluid reservoir surface, wherein the first layer of fabric contains lyocell fibers;
(ii) a second layer of fabric, wherein the second layer of fabric contains lyocell fibers; and
(iii) at least one joining mechanism,
wherein the at least one joining mechanism joins the first layer of fabric to the second layer of fabric; and
wherein the wound care device transports wound fluid uni-directionally from the wound contact surface to the wound fluid reservoir surface upon exposure to a wound site;
(b) placing the wound contact surface of the wound care device in contact with the wound site; and
(c) allowing the wound care device to transport wound fluid uni-directionally from the wound contact surface to the wound fluid reservoir surface.
